(19) United States
(12) Patent Application Publication
(10) Pub. No.: US 2008/0009889 A1
(43) Pub. Date: Jan. 10, 2008

(54) Conduit cuff

(76) Inventors: James L. Pokorney, Northfield, MN (US); Kemal Schankereli, Stillwater, MN (US)

Correspondence Address: Jim Pokorney, 303 Washington, Northfield, MN, 55057, US

(21) Appl. No.: 11/825,186

(22) Filed: Jul. 5, 2007

Related U.S. Application Data
(60) Provisional application No. 60/818,661, filed on Jul. 5, 2006.
(60) Provisional application No. 60/818,662, filed on Jul. 5, 2006.

(51) Int. Cl. A61M 39/00

(52) U.S. Cl. 606/155; 604/533

(57) ABSTRACT

The primary object of the present invention is to provide an improved anastomotic connection between a conduit and a vessel by use of a cuff attached to one end of the conduit. By using a cuff, the conduit can be connected to a vessel without first cutting the vessel and without clamping the underlying vessel. In one embodiment of the invention, the cuff is contoured in the shape of a saddle to provide a connection cuff that contours to the cylindrical shape of the underlying vessel. The invention provides methods to connect the conduit to vessel without having the sutures located at the intersection of conduit and vessel, thereby minimizing the possibility that a secondary cutting operation of the vessel will also cutting the sutures. In one embodiment of the invention, a transition in compliance between the generally stiffer conduit and the generally softer vessel is accomplished by forming a compliance transitioning interface at the junction of the conduit and cuff.

This application claims priority from provisional patent application U.S. Ser. Nos. 60/818,661 and 60,818,662 filed by inventors on Jul. 5, 2006.

### BACKGROUND

1. Field of Invention

This invention relates to an improved conduit cuff and an improved cuff attachment to a body vessel.

2. Applicant Patent Application 20050149093

A less invasive means invented by applicant to implant a valve bypass graft is described in U.S. Patent Application 20050149093 which is hereby incorporated by reference in its entirety. This invention relates to an implant, implant tools, and an implant technique for the interposition of an extracardiac conduit between the left ventricle of a beating heart and the aorta to form an alternative one-way blood pathway thereby bypassing the native diseased aortic valve.

Although this prior invention provides key enabling technologies that will allow mainstream use of the valve bypass graft procedure, an improved conduit cuff design is needed to make the procedure safer and more effective. The improved design described herein could be used in many surgical applications.

### OBJECTS AND ADVANTAGES

The primary object of the present invention is to provide an improved anastomotic connection between a conduit and a vessel by use of a cuff attached to one end of the conduit. The invention is an improvement over prior art because embodiments of the invention:
allow connection of conduit to vessel without first cutting the vessel
allow connection of conduit to vessel without clamping the underlying vessel
provide a connection cuff that contours the cylindrical shape of the underlying vessel
provide a connection of conduit to vessel without having the sutures located at the intersection of conduit and vessel, thereby minimizing the possibility that a secondary cutting operation of the vessel might also cutt the sutures
provide a connection cuff that is reinforced with a wire ring to ensure a leak free connection
allow for the use of a hemostatic agent or biocompatible glue at the interface of the cuff and underlying vessel
allow for an angular connection between conduit and vessel
provide a transition in compliance between the generally stiffer conduit and the generally softer vessel
provide a device and method for converting a slit in a vessel into a generally oval shaped blood flow opening by employing a cuff with a pre-stressed internal shaping ring

The above mentioned objects and advantages of this invention will become apparent from the following description taken in connection with the accompanying drawings, wherein is set forth by way of illustration and example, preferred embodiments of this invention.

### DESCRIPTION OF DRAWING FIGURES

In the drawings, closely related figures have the same number but different alphabetic prefixes.

FIG. 1 shows a perspective view of prior art

FIG. 2 shows a side view of prior art.

FIG. 3 shows a front, side, and top view of one embodiment of the invention.

FIG. 4 shows a front, side, and top view of one embodiment of the invention.

FIGS. 5A-B shows a side view and a top view of prior art.

FIGS. 6A-B shows a side view and a top view of one embodiment of the invention.

FIG. 7 shows a front, side, and top view of a ring.

FIG. 8 shows a front and side view of one embodiment of invention using ring shown in FIG. 7.

FIG. 9 shows one embodiment of the invention.

FIG. 10 shows one embodiment of the invention.

FIG. 11 shows one embodiment of the invention.

FIG. 12 shows one embodiment of the invention.

FIG. 13 shows one embodiment of the invention.

FIG. 14A-B shows a top view of a Cuff embodiment in two different states.

FIG. 14A-B shows a top view of a Cuff embodiment in two different states.

FIG. 16A-C shows an embodiment of a Cuff/Conduit and a Cuff Tool.

FIGS. 17-21 shows embodiments of a Cuff/Conduit and Cuff Tool in side views and top views of an associated Cuff/Ring assembly.

### DEFINITIONS

The term “vessel” when used herein in relation to the Conduit/Cuff inventions refers to any artery, vein, passageway, or organ in the body. As examples, vessel could be the aorta or the heart.

### Invention Description

In this invention, a new and unique devices and methods to attach a vascular graft or conduit to a body vessel are described.

To those knowledgeable in the art of surgery, prosthetic vascular conduits are attached to native arteries or veins using direct edge to edge suturing methods in a blood free field. To perform this connection, or anastomosis, the native vessel needs to be accessed and opened. Either blood flow needs to be stopped by clamping upstream of the attachment site or a special Side Bite Clamp 2, as shown in FIG. 1, is used to divert flow around the Attachment Site 4. A side bite is preferable if the procedure is anticipated to be done without cardiopulmonary bypass because distal flow is allowed during the procedure. In either case though, the Vessel 6 needs to be manipulated and eventually pinched. This action can loosen calcified or atherosclerotic plaque which can then migrate downstream, occluding smaller vessels. In current practice, once flow is stopped at the selected connection site, the Vessel 6 is cut open and then attachment to the Conduit 8 can commence. In summary, to connect a large diameter conduit to a vessel, the surgical steps, in order, are:
1. Stop Flow
2. Open Vessel
3. Attach Conduit

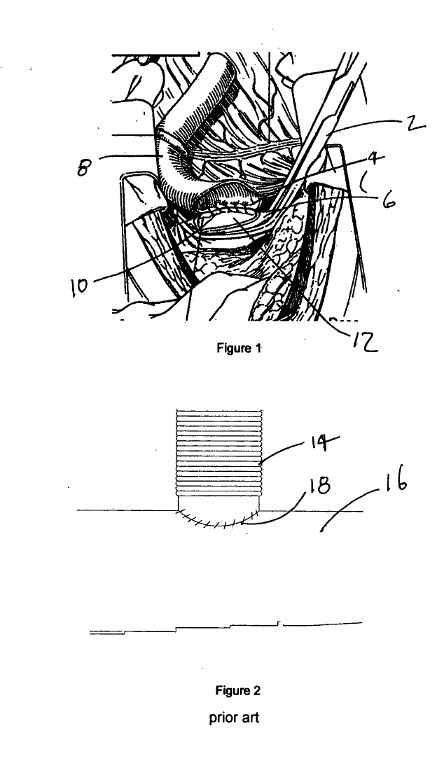

The prosthetic Conduit 8 is attached directly to the Vessel 6 by means of Sutures 10 such that the edge of the Conduit 8 abuts the edge of the Hole 12 made in the Vessel 6. A more simplistic drawing showing the prior art end to side attachment method is shown in FIG. 2. In this method, the cylindrical Conduit 14 is sewn to the cylindrical native Vessel 16 using Sutures 18 at the intersection.

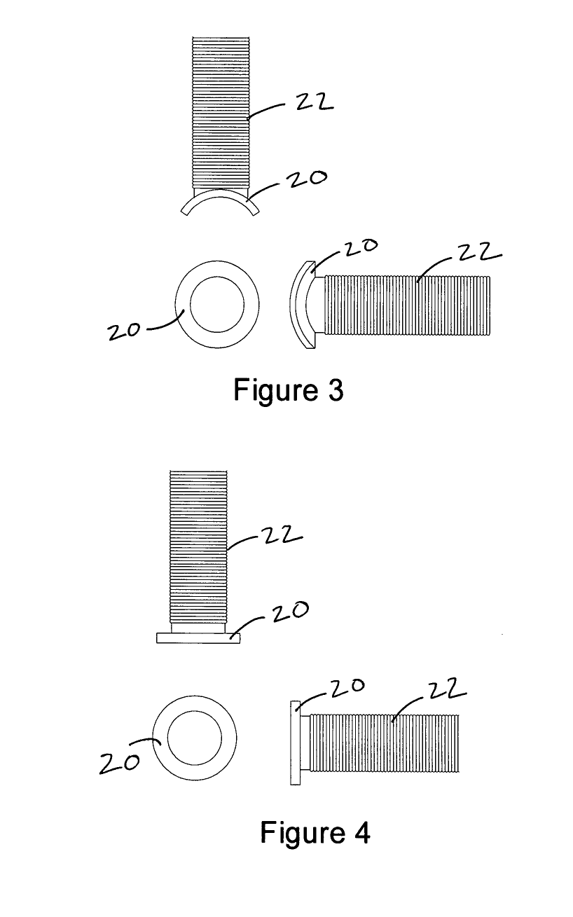

Typical embodiments of the new invention is shown in FIGS. 3 and 4. The device is modified to have an attachment Cuff 20 attached to one end of a Conduit 22. The Cuff 20 can be saddle shaped to better fit the radius of the native vessel or it could be formed in a more planar “top hat” fashion as shown in FIG. 4 since the vessel is compliant and can be shaped to the cuff at attachment.

In prior art designs, the conduit is attached to the vessel directly using sutures such that the sutures intimately connect the free edge of the conduit with the cut edge of the native vessel using a technique commonly referred as “edge to edge” suturing. This technique requires that the cut vessel wall be exposed and accessible for the surgeon to insert the sutures. This requires a blood free field, necessitating a localized stoppage of blood flow.

In the new invention, an open surgery edge to edge connection is not performed. The flow is not stopped because the attachment is made before the vessel is cut open. Here are the attachment steps in order.
1. Do not stop or partially occlude flow
2. Attach Conduit
3. Open Vessel

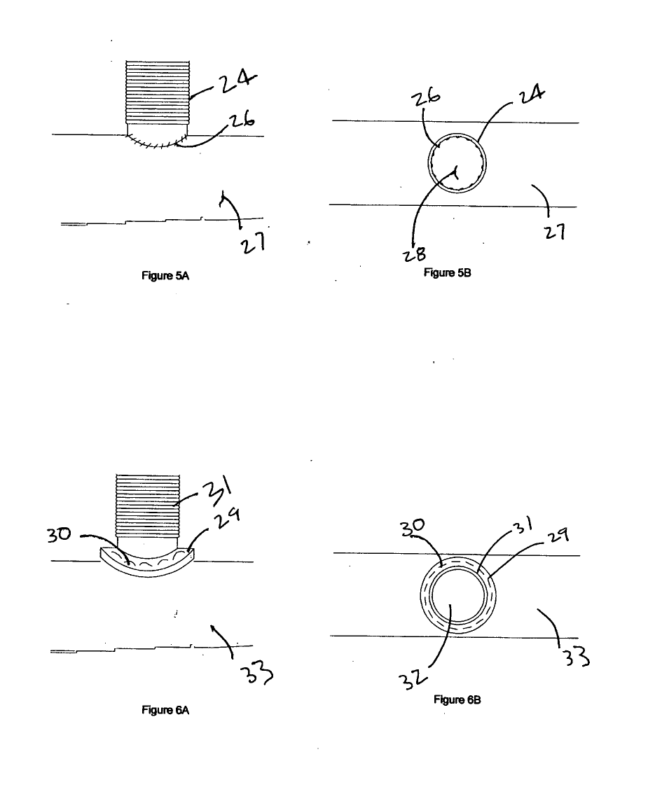

The key to this design is the use of a cuff attached to the conduit. The cuff, which could be composed of polyester, Teflon, or some other biocompatible material, allows the surgeon to sew the conduit to the vessel wall without interfering with the flow pathway. The cuff provides a terrific blood barrier and the suture attachments are not in the direct flow path compared to prior art. In FIGS. 5A-B is shown the prior art Conduit 24 both in side view and in top view showing how the Sutures 26 are in the Blood Pathway 28 entering Vessel 27. In FIGS. 6A-B is shown the new invention in side view and top view showing the Cuff 29 attached to Conduit 31. Cuff 29 is shaped to have an outside diameter larger than the outside diameter of Conduit 31. In this embodiment of the invention, Cuff 29 outside diameter is 8 millimeters larger the the Conduit 31 outside diamet but could range, in different embodiments of the invention, between 2 and 14 millimeters larger. Because the Cuff 29 is larger in diameter than the Conduit 31, Sutures 30 are located a distance away from the Blood Pathway 32 entering Vessel 33. This distance is typically a minimum of about 1 millimeter or so. Also, the sutures are not in the pathway of a cutter tool if it is inserted through the Conduit 31 as anticipated in patent application U.S. Ser. No. 60/818,664. A sharp cutter inserted into the conduit can cut an access hole nearly identical in size to the inside diameter of the conduit without fear of cutting into the sutures since the sutures are not located at the intersection of the conduit and vessel as is typical in prior art. The ratio of the cuff outside diameter to the conduit outside diameter should be 1.1 or larger to allow sufficient room on the cuff to suture into a vessel without interfering with any cut hole through the conduit.

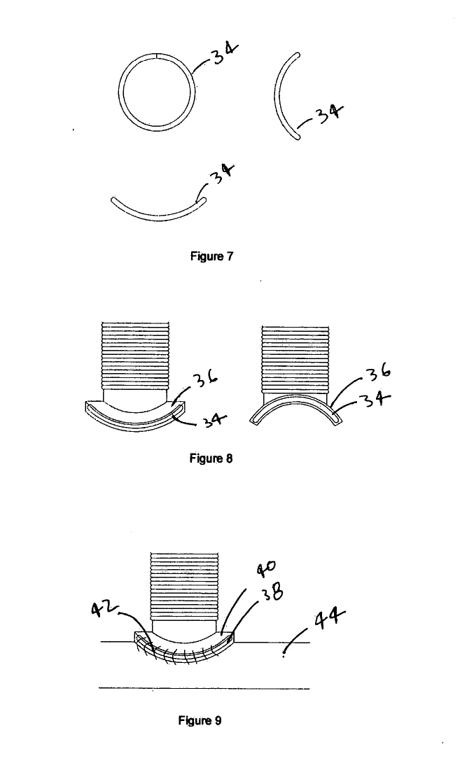

To maintain the cuff shape and to facilitate a blood tight seal to the vessel surface, the Cuff can be assembled with a circular or saddle shaped wireform built into the Cuff. The wireform is constructed of stainless steel, nitinol, polycarbonate, or some other rigid biocompatible material capable of maintaining a pre-defined form. In FIG. 7 is a drawing showing a saddle shaped Ring 34 in front, side and top view. In FIG. 8 is shown the same Ring 34 inserted into a Cuff 36 shown in front and side view. The wire shape is formed by wrapping a stainless steel wire around a forming mandrel and then applying a heat treatment to hold the wrapped shaped. The wire can be wrapped around the mandrel a minimum of one rotation. The ends of the wire could be attached to each other to form a continuous ring or could be left unattached. Other wireform shapes could be employed to create a shape compatible with the underlying vessel.

FIG. 9 shows a Wireform 38 within a Cuff 40. A suture 42 looped over the Wireform 38 when attaching the Cuff 40 to an underlying Vessel 44 will transfer its retention force along the length of the Wireform 38. If multiple sutures are placed over the Wireform 38, this force distribution from suture to suture along the Wireform 38 will encourage a blood tight seal along the surface of the Cuff 40 in contact with the Vessel 44. Therefore, the Wireform 38 will improve the Cuff 40 to Vessel 44 seal.

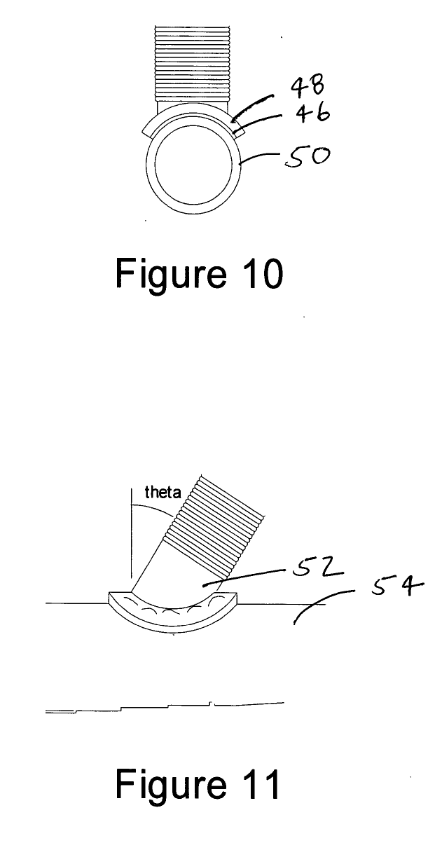

One common problem encountered when attaching vascular prosthesis onto autologous vessels relates to control of bleeding at the anastomotic site. Since sutures de facto create holes in the cuff as well as the underlying adventitia inhibition of bleeding must be considered. Because the cuff is not directly in the blood pathway, it is possible to place a Surface Modifier 46 between a Cuff 48 and Vessel Surface 50 as shown in FIG. 10.

This surface modifier could promote hemostasis in anticipation of cutting the hole in the vessel or it could promote long term healing of the cuff into the vessel. A similar approach can easily be envisioned for the attachment of the cuff to the apex of the heart where similar issues are apparent.

One particularly useful modification relates to the incorporation of hemostatic agents within as well as onto the cuff surface. In this example the hemostatic agent reacts with blood normally present in the surgical field to coagulate blood on and under the cuff to form a leak proof attachment. Examples of hemostatic agents that can be considered for this application include materials such as potassium bromide (KBr), collagen and chitosan.

Foreign materials often create strong inflammatory responses when implanted in the body. To mitigate these reactions at the attachment point of the cuff to the vascular adventitia or at the apex of the heart anti-inflammatory agents can be infused into the cuff and made to slowly release and inhibit the inflammatory responses. One example of a useful anti-inflammatory agent is a material such as glycosaminoglycan.

As shown in FIG. 11, a Conduit/Cuff Assembly 52 can be designed to align with the underlying Vessel 54 at an angle theta other than perpendicular. For instance the angle could be between 30 and 60 degrees to facilitate preferential blood flow in one direction in the underlying Vessel 54.

It is known to those in the art of artificial blood conduits that a pseudointimal lining eventually covers the inside surface of textile conduit implants. Also, it is known that at the juncture of the conduit to the native vessel, it is likely that the pseudointima lining grows thicker and can restrict blood flow. People knowledgeable in the art attribute this undesirable entimal hyperplasia to the fact that the radial compliance, or stiffness, of the conduit is different than the compliance of the native vessel. Compliance matching has been closely studied and has been indicated as one factor in mitigating anastomotic line hyperplasia. Thus, one effective criteria for improving overall device performance relates to minimizing hyperplasia by matching the radial compliance at the anastomotic site. Designs whereby gradual transitions from elastic to stiff are apparent at vessel attachment sites are therefore preferred to those in which abrupt transitions occur.

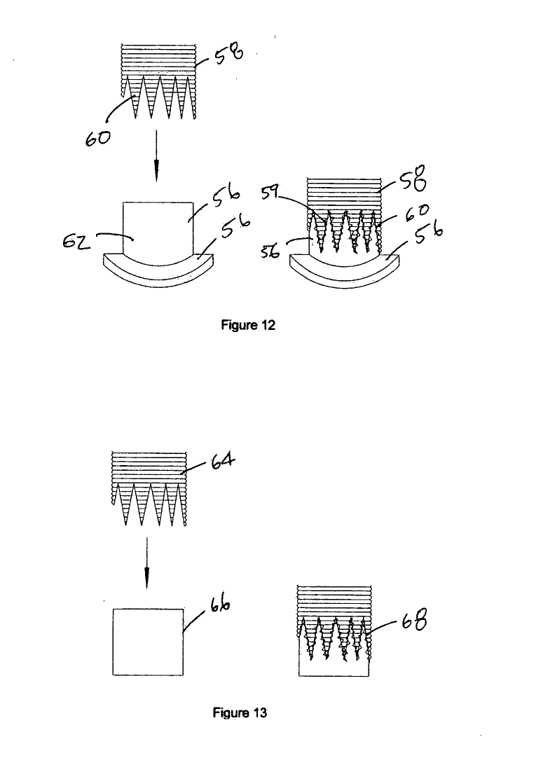

In one embodiment of the Cuff/Conduit design as shown in FIG. 12, the Cuff 56 that is sewn to the vessel is composed of a more compliant, stretchier material compared to a Conduit 58. The Cuff 56 can stretch radially as required if the underlying vessel is stretched. The Cuff 56 is designed to be tubular in shape and can be made from polyester or some other stretchy biocompatible material. One end of the Cuff 56 is rolled up or otherwise shaped to form the larger diameter cuff shape. The remainder of the Cuff 56 remains tubular in shape to allow attachment to the tubular shaped Conduit 58. The Conduit 58 is typically less stretchy, or less compliant, than the Cuff 56.

It is at this transition that a major shift in compliance occurs. To smooth out this necessary transition from more compliant Cuff 56 to less compliant Conduit 58, the conduit material is cut in a zigzag or some other curvilinear pattern such that the amount of conduit material is gradually increased from the cuff end when measured along the long axis. This design creates multiple fingers or Appendages 60 on the end of the Conduit 58 that are generally thinner near the cuff end of the conduit. The perimeter or length of these appendages would be substantially longer than a minimum perimeter of piD, D equaling the Diameter of the conduit. For instance, the perimeter measurement of the curvilinear end of the conduit could be 1.5 piD or greater. Once the Conduit 58 is prepared with such a transition, it can be joined to the Cuff 56. The Cuff 56 is connected to the Conduit 58 by Suture 59, adhesive, or some other means that firmly adheres the conduit Appendages 60 to the Cuff Tubular Section 62. When such an assembly is subjected to internal fluid pressure, a cross section of the assembly near the more compliant Cuff 56 will expand more and a cross section near the less compliant Conduit 58 will expand less. In a cross section where there is a combination of Cuff and Conduit material, the amount of stretch or compliance will vary depending on the ratio of high compliance Cuff material to low compliance Conduit material.

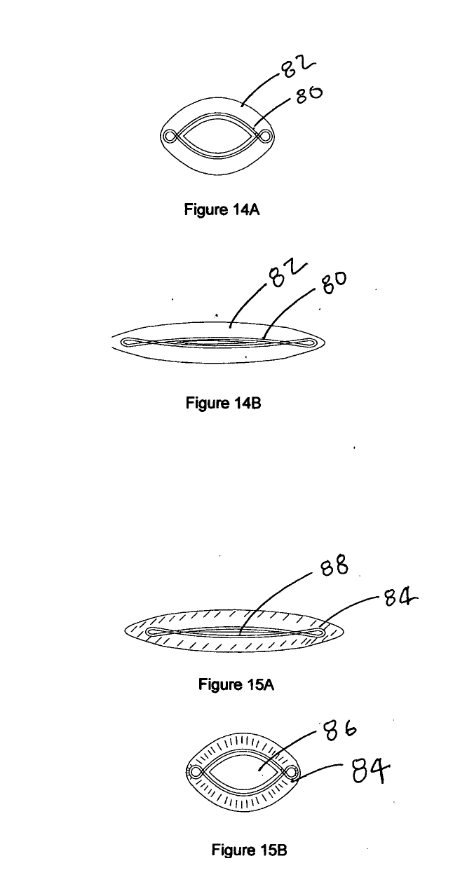

Another embodiment of this compliance transition invention is shown in FIG. 13. The larger diameter Cuff element can be eliminated such that a high compliance tubular Conduit 64 is connected to a low compliance tubular Conduit 66 such that the resultant Hybrid Conduit 68 is of a generally isodiametric diameter with a compliance transition near one or both ends to facilitate a smooth compliance transition to native vessels.

The invention embodiments disclosed so far describes generally circular cuffs and conduits intended to be used in conjunction with removing a circular like piece of tissue about the same size and shape of the attached conduit using a blade or cutting drum as described in USPTO patent application 20050149093. To those knowledgeable in the art of surgery, it is also a good goal to minimize the possibility of allowing loose pieces to enter the blood stream. Therefore, an alternative idea to create a sufficient access hole in a vessel is to form the hole by cutting a slit without removing any tissue. After cutting, the slit is transformed into an oval by using energy stored in the conduit cuff.

In a single slit design, the cuff contains an elastic shaping member within the cuff. A drawing of such a cuff is shown in FIGS. 14A-B. An elastic spring-like Ring 80 is shaped to be normally circular as shown in FIG. 14A but can be compressed to be more slit likel as shown in FIG. 14B. The Ring 80 can be composed of one or more spring elements composed of nitinol, stainless steel, or some other appropriate biocompatible elastic material. A top view of a Cuff 82 with Ring 80 within is shown in FIGS. 14A-B. The conduit is not shown. In FIG. 14A a Ring/Cuff Assembly 84 is shown in its normal, uncompressed state. In FIG. 14B the Ring Cuff Assembly 84 is shown in a compressed state.

In FIG. 15A, the Ring/Cuff Assembly in its compressed state is shown sewn to an artery. In FIG. 15B, the same assembly is shown in its normal, uncompressed state. Note the enlarged Hole 86 in FIG. 15B compared to the small slit like Opening 88 in FIG. 15A.

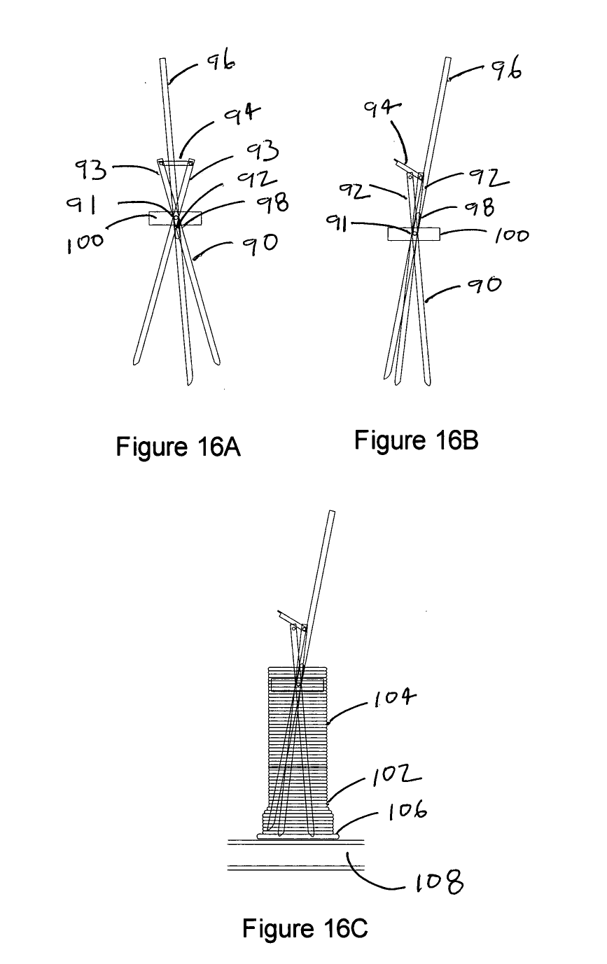

In use, the Ring/Cuff Assembly 84 is held in a compressed state with a Restraining/Cutting Tool 90. This Tool is shown in FIGS. 16A-B. The Tool 90 is scissors like in design. A Clevis Pin 91 holds three shafts together about a common point of rotation. Two Restraining Shafts 92 can he positioned such that their tips are a fixed distance apart by engaging a Locking Bar 94. The Restraining Shafts 92 can be released and allow to swing independently by releasing Locking Bar 94. Also attached to Clevis Pin 91 is Cutting Shaft 96. This Shaft is attached to the two Restraining Shafts 92 using a Slot 98 cut into the Shaft 96. This Slot 98 allows the Cutting Shaft to move longitudinally relative to the Restraining Shafts 92. A circular Plug 100 is attached around the Clevis Pin 91 region of Tool 90.

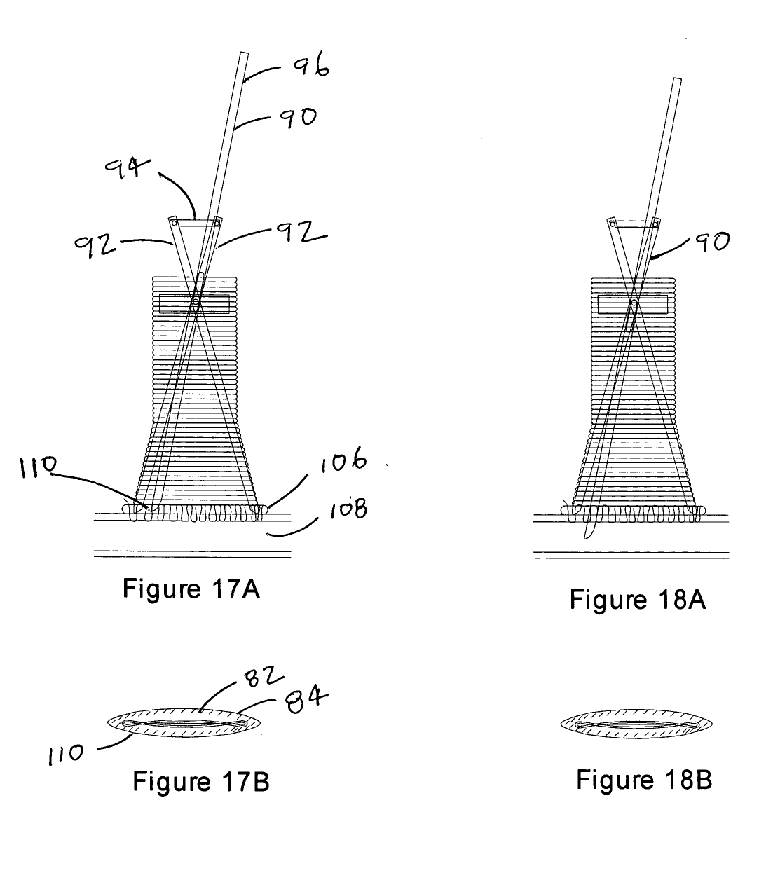

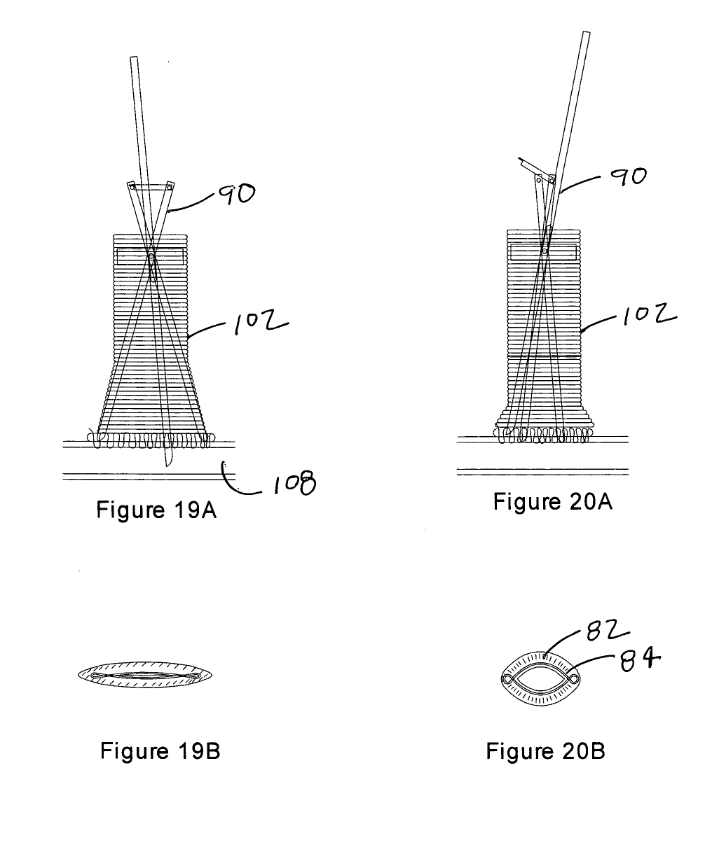

In use, Tool 90 is inserted in a Conduit/Cuff Assembly 102 as shown in FIG. 16C, and FIGS. 17 through 21. When inserted, the Plug 100 makes a near blood tight seal with the Conduit 104. A Cuff 106 can be sewn to an underlying Vessel 108 using Sutures 110. Before sewing, the Retraining Shafts are aligned with the long axis of the Ring/Cuff Assembly 84 and locked into place using the Lock Bar 94. This action spreads the Ring 80 within Cuff 92 to its deformed, slit-like shape. After the Cuff 82 is sewn into place, the Cutting Shaft is advanced toward the Vessel 108 along Slot 98 until it is inserted into the Vessel 108 as shown in FIG. 18A. The Cutting Shaft 96 is then rotated about Clevis Pin 91 to create a slit in Vessel 108 within Cuff 82. After the slit is made, the Locking Bar 94 is released, the Restraining Shafts are moved together, and the Restraining Cuff Tool 90 is removed from the Conduit/Cuff Assembly 102.

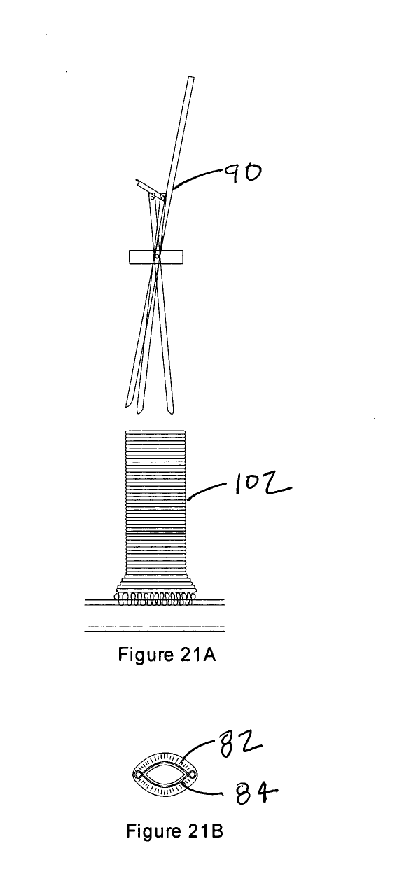

Once the Tool 90 is removed, the Cuff, with internal Ring 80 now free to reshape to its lowest energy state, creates a more circular shaped opening in Vessel 108 compared to the slit cut. This larger flow area is shown in FIGS. 20B and 21B. Since the underlying Vessel 108 is compliant, the restraining stresses generated within the vessel tissue are not sufficient to stop the slit from enlarging sufficient to carry adequate blood flow.

By employing this design, adequate flow can be achieved and no loose tissue pieces are generated.

### SUMMARY

The reader will see that the invention is an improvement over prior art. In particular, because a cuff is attached to a conduit, the conduit can be connected to the vessel without first cutting the vessel and without clamping the underlying vessel. In one embodiment of the invention, the cuff is contoured in the shape of a saddle to provide a connection cuff that contours to the cylindrical shape of the underlying vessel. The invention provides a method to connect the conduit to vessel without having the sutures located at the intersection of conduit and vessel, thereby minimizing the possibility of a secondary cutting operation of vessel also cutting the sutures. Also, the cuff can be constructed with a reinforcing wire ring to ensure a leak free connection. If sutures alone are not sufficient, or perhaps if sutures are not used at all, the invention allows for the use of a hemostatic agent or biocompatible glue at the interface of the cuff and underlying vessel to create a good biocompatible connection. In some situations, it may be useful to use the cuff but still allow for an angular connection between conduit and vessel. This is possible with a pre-configured angular conduit/cuff assembly. In one embodiment of the invention, a transition in compliance between the generally stiffer conduit and the generally softer vessel is accomplished by forming a compliance transitioning interface at the junction of the conduit and cuff.

Although the description above contains many specifications, these should not be construed as limiting the scope of the invention but as merely providing illustrations of the presently preferred embodiment of this invention. Thus, the scope of the invention should be determined by the appended claims and their legal equivalents rather than by the examples given.

1. A conduit cuff assembly consisting of:
a) a cuff having a first surface and a second surface,
b) a conduit having an open end,
c) a biocompatible agent,
d) said open end of said conduit attached to said first surface of said cuff,
e) said biocompatible agent applied to said second surface of said cuff.

2. A conduit cuff assembly consisting of:
a) a cuff having an inside diameter and an outside diameter,
b) a conduit having an inside diameter and an outside diameter,
c) the ratio of said inside diameter of said cuff to said inside diameter of said conduit is between 0.95 and 1.05, and
d) the ratio of said outside diameter of said cuff to said outside diameter of said conduit is greater than 1.1

3. A blood carrying conduit assembly consisting of:
a) a first blood carrying conduit having a material compliance and an open end,
b) a second blood carrying conduit having a material compliance, said material compliance is less than said material compliance of said first blood carrying conduit, and said second blood carrying conduit having an open end, said open end having a perimeter, said perimeter is larger than the circumference of said second blood carrying conduit, and
c) connecting means for joining said open end of said first blood carrying conduit with said perimeter of said open end of said blood carrying second conduit to form a blood carrying conduit with a material compliance that transitions along its length from a higher compliance to a lower compliance.
